(19) United States
(12) Patent Application Publication
(10) Pub. No.: US 2023/0363761 A1
(43) Pub. Date: Nov. 16, 2023

(54) METHOD AND SYSTEM OF REDUCING LIMB ISCHEMIA

(71) Applicant: ABIOMED, Inc., Danvers, MA (US)

(72) Inventors: Robert Swierczek, Danvers, MA (US); Claudia Mourran, Danvers, MA (US)

(73) Assignee: ABIOMED, Inc., Danvers, MA (US)

(21) Appl. No.: 18/195,695

(22) Filed: May 10, 2023

Related U.S. Application Data
(60) Provisional application No. 63/340,358, filed on May 10, 2022.

(51) Int. Cl. A61B 17/12

(52) U.S. Cl. CPC A61B 17/12036; A61B 17/12109; A61B 17/12136; A61B 2017/12127

(57) ABSTRACT

A device and method are provided for minimizing and/or preventing limb ischemia, such as when a medical device is inserted into a patient’s vasculature that may occlude the vessel. The device may include a tubular member and one or more radially expandable features, such as balloons and/or hydrostatic skeletons, configured to be expanded at a target site, allowing blood to flow around and/or through the radially expandable features.

### CROSS-REFERENCE TO RELATED APPLICATIONS

The present application claims priority to US 63/340,358, filed May 10, 2022, the entirety of which is incorporated by reference herein in its entirety.

### TECHNICAL FIELD

The present disclosure relates to methods, devices, and systems used in the prevention of limb ischemia, such as that resulting from use of a mechanical circulatory support device.

### BACKGROUND

Limb ischemia is a rapid and sudden decrease in limb perfusion often threatening limb viability. It may occur as a result of blockage of blood due to an indwelling sheath and/or catheter, local occlusion (e.g., atherosclerotic narrowing), and/or continuous occlusion resulting from small vessels and/or large sheaths. It may also occur as a result of a closure issue.

Limb ischemia is associated with mortality, and some literature indicates the rate of limb ischemia may be high. As such, in addition to negatively impacting the patient, it may also negatively impact ongoing clinical trials.

### BRIEF SUMMARY

In various aspects, a device may be provided. The device may include a tubular member. The device may include one or more radially expandable features disposed on an outer surface of the tubular member. The one or more radially expandable features may be configured to be selectively expanded from a collapsed configuration to an expanded configuration to allow blood within a blood vessel of a patient to flow from a point upstream of the one or more radially expandable features, around and/or through the one or more radially expandable features, and to a point downstream of the one or more radially expandable features.

The one or more radially expandable features may include one or more balloons. The one or more radially expandable features may extend along a portion of a length of the tubular member. Each radially expandable feature may have an identical axial length. In some embodiments, at least one of the one or more radially expandable features may have a different axial length than another of the one or more radially expandable features.

The one or more radially expandable features may include a plurality of radially expandable features. Each radially expandable feature may be separated circumferentially from an adjacent radially expandable feature by an identical distance. In some embodiments, each of the plurality of radially expandable features may be separated circumferentially from an adjacent radially expandable feature. A circumferential separation distance of a first adjacent pair of adjacent radially expandable features may be different from a circumferential separation distance of a second adjacent paid of adjacent radially expandable features.

A central axis of each radially expandable features may be parallel to a central axis of the tubular member. At least one radially expandable feature may be disposed in a helical pattern on the outer surface of the tubular member. At least one radially expandable feature may have an oval or circular cross-sectional shape. At least one radially expandable feature may have a polygonal cross-sectional shape.

Each of the radially expandable feature(s) may be fluidly connected to a fluid source. Each of the radially expandable feature(s) may be operably connected to a connector. The radially expandable feature(s) may include a hydrostatic skeleton.

In various aspects, a device may be provided. The device may include a tubular member and may include a hydrostatic skeleton coupled to the tubular member. The hydrostatic skeleton may be configured to be selectively inflated (“expanded”) or deflated (“compressed”). The hydrostatic skeleton may be configured to be expanded from a collapsed configuration to an expanded configuration to create at least a first channel for allowing blood to flow from a point upstream of the hydrostatic skeleton, through the first channel, to a point downstream of the hydrostatic skeleton.

The hydrostatic skeleton may be configured to be selectively inflated or deflated to create at least the first channel and a second channel. The cross-sectional area of the first channel and a cross-sectional area of the second channel may be identical. The cross-sectional area of the first channel and a cross-sectional area of the second channel may be different. A cross-sectional area of the first channel may be controlled separately from the cross-sectional area of the second channel. The tubular member may be configured to slidably receive a second tubular member. The device may include a valve at a proximal end of the tubular member, the valve being operably coupled to the second channel.

The hydrostatic skeleton may extend fully around a circumference of the tubular member. The hydrostatic skeleton may extend only partially around a circumference of the tubular member. The hydrostatic skeleton may be coupled to a distal end of the tubular member. At least a portion of the hydrostatic skeleton may extend along an axial length of the tubular member. A diameter of the first channel may increases when the hydrostatic skeleton is inflated. The hydrostatic skeleton may include a plurality of annular rings.

In various aspects, a method may be provided. The method may include positioning a tubular member within a blood vessel, the tubular member having an outer surface and at least one radially expandable member coupled to the outer surface. The method may include expanding the least one radially expandable feature from a collapsed configuration to an expanded configuration to allow blood to flow from a point upstream of the at least one radially expandable feature, around and/or through (e.g., through an opening or channel created by the feature) the at least one radially expandable feature, and to a point downstream of the at least one radially expandable feature.

The step of expanding the at least one radially expandable feature may include expanding a portion of the blood vessel around the at least one radially expandable feature.

The step of expanding from a collapsed configuration to an expanded configuration may include expanding all of the plurality of radially expandable features simultaneously. The step of expanding from a collapsed configuration to an expanded configuration may include selectively expanding fewer than all of the plurality of radially expandable features.

The step of expanding from a collapsed configuration to an expanded configuration may occur automatically based on received information. The received information may be a determination that an ischemic event is occurring, or may be occurring. The received information may be a value from a sensor.

The method may include measuring a pressure within the blood vessel, such as upstream and/or downstream from the from the radially expandable feature(s). The at least one radially expandable feature may include a balloon. The at least one radially expandable feature may include a hydrostatic skeleton.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1A is an illustration of a device positioned within a blood vessel.

FIG. 1B illustrates a cross-section of the device of FIG. 1.

FIG. 1C illustrates a cross-section of another embodiment of a device according to the present disclosure.

FIG. 2A is an illustration of a device positioned within a blood vessel.

FIGS. 2B-2C are illustrations of various embodiments of cross-sections of the device of FIG. 2A.

FIG. 2D is an illustration of another embodiment of a device position within a blood vessel.

FIGS. 2E-2F are illustration of various embodiments of cross-sections of the device of FIG. 2D.

FIG. 2G is an illustration of another embodiment of a device position within a blood vessel.

FIG. 3 is a flowchart of a method.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or”, as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

As is known, limb ischemia is a rapid and sudden decrease in a patient’s limb perfusion, which may occur because of a blockage of blood due to an indwelling sheath and/or catheter, a local occlusion (e.g., an atherosclerotic narrowing), and/or a continuous occlusion resulting for small vessels and/or large sheaths. Limb ischemia also may occur as a result as a result of a closure device.

In some instances, to address limb ischemia, a small diameter catheter may be inserted distally to provide antegrade flow to the ischemic limb. For example, the catheter may be inserted either in the dorsalis pedis artery, or the posterior tibial. As will be appreciated, the antegrade flow may be introduced at any suitable time (e.g., at the time of initial ECMO cannulation or implantation of a mechanical circulatory support device or at a later time, such as after an ischemia is discovered). In some instances, conventional solutions may not be available to all patients. For example, some patients may not have a large enough dorsalis pedis artery or posterior tibial for a catheter to be introduced.

The inventors have recognized that improved patient outcomes may result when ischemic events are quickly identified and addressed. The inventors have also recognized the benefit of promoting and providing distal limb perfusion to patients whose arteries are occluded by expanding the vasculature around at least a portion of a fully introduced cannula (e.g., introducer sheath). For example, as described herein, expanding the vasculature around at least a portion of the cannula may allow for blood flow (e.g., retrograde flow) to go around and past the cannula. In this regard, the disclosed devices and methods may address the unmet needs of currently existing distal limb perfusion techniques. The disclosed devices and methods also may reduce the number of insertion sites in a patient, which may reduce the risk of infection, sepsis, bleeding, hematoma, ischemia, and pseudoaneurysm.

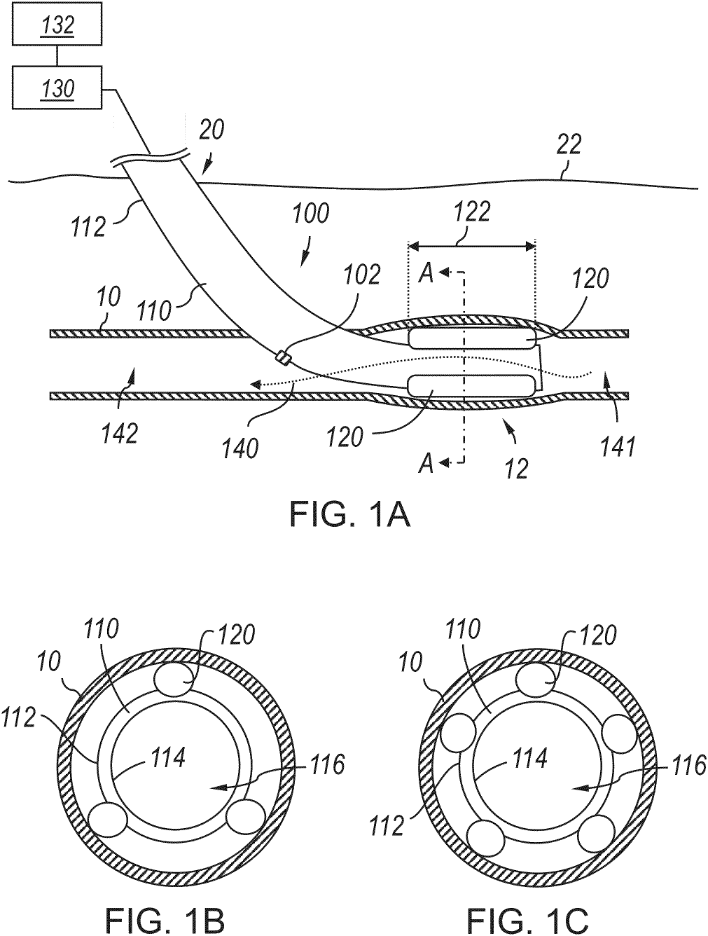

In various aspects, a device and method for promoting and providing distal limb perfusion, and reducing limb ischemia, may be provided. Referring to FIG. 1A, the device 100 may include a tubular member 110, which may be inserted into the patient’s vasculature. For example, the tubular member may be designed to be inserted at an access point 20, through skin 22, and into a blood vessel 10, as shown in FIG. 1A. In some embodiments, the tubular member may be a sheath, a cannula, and/or a catheter, although other suitable tubular members may be used. The tubular member may be composed of any appropriate material, such as nitinol, a medical grade polymer (such as a polyurethane), and/or combinations thereof. The tubular member may be formed of other suitable materials in other embodiments.

As described herein, the tubular member may be configured to selectively expand the at least a portion of the patient’s vasculature around the tubular member. In that regard, as shown in FIGS. 1A-1C, the tubular member may include one or more features 120 configured to expand radially, to move at least a portion 12 of the vessel 10 away from the tubular member to allow blood to flow 140 from a point 141 upstream of the feature(s), between the tubular member and the vessel, and to a point 142 downstream of the feature(s). For example, in some embodiments, the features may have a collapsed configuration, such as when the tubular member is inserted into the patient, and an expanded configuration, such as that shown in FIG. 1A where the one or more features are expanded to move the portion 12 of the vessel away from the sheath. As will be appreciated, a diameter (e.g., a distance from the center of the tubular member to the outer most portion of the expandable feature) of the tubular member in larger when the features are in the expanded configuration than when the features are in the collapsed configuration.

The feature may be composed of any appropriate material, such as nitinol, a medical grade polymer (such as a polyurethane), or other suitable materials. As will be appreciated, the feature may be formed of an expandable material. In some embodiments, the features may each include a balloon that may be inflatable, such as via the controlled addition of a fluid from a fluid source 132. In some embodiments, the device may include a controller 130 that selectively expands and/or contracts the features by adding and/or removing the fluid from the feature. In some embodiments the fluid may be saline, although other suitable flids, such as gas, may be used. In some embodiments, the fluid may pass through one or more lumens (not shown) in the tubular member. In that regard, the fluid source may be fluidly coupled to the features via the one of more lumens in the tubular member.

As shown in FIGS. 1A-1C, the tubular member may include a plurality of features 120 extending around a circumference of the tubular member. Referring to FIG. 1B, the features may be configured such that each feature 120 may be disposed on an outer surface 112 of the tubular member 110. An inner surface 114 of the tubular member may define a lumen 116 extending therethrough.

The number of features may vary. As seen in FIG. 1B, the device may have three features. In FIG. 1C, the device is shown as having five features. However, any number of features may be present. For example, in some embodiments, the tubular member may have one or more expandable features.

In various embodiments, the arrangement of the radially expandable feature(s) may vary. In some embodiments, the one or more radially expandable features may extend along a portion of an axial length 122 of the tubular member. In some embodiments, each radially expandable feature may have an identical axial length. In some embodiments, the radially expandable features may not be identical. For example, at least one radially expandable feature may have a different axial length than another. In another example, at least one radially expandable feature may have a diameter that is different than that of another radially expandable feature. In some embodiments, a central axis of each radially expandable features may be parallel to a central axis of the tubular member. In some embodiments, the radially expandable feature(s) may be disposed in a helical pattern (or partial helical pattern) on the outer surface of the tubular member.

As will be understood, in some embodiments, radially expanding features may be separated along an axial length of the tubular member, not just simply circumferentially separated from one another.

The device may include a plurality of radially expandable features. The features may be arranged in various ways relative to each other. For example, each radially expandable features may be separated circumferentially from an adjacent radially expandable feature by an identical distance. In some embodiments, a circumferential separation distance of an adjacent pair of the plurality of adjacent radially expandable features is different from a circumferential separation distance of a different adjacent pair of the plurality of adj acent radially expandable features.

The shape of the radially expandable features may vary. The radially expandable feature(s) may have an oval or circular cross-sectional shape, a polygonal cross-sectional shape, or a combination thereof. Each feature may have an identical cross-sectional shape. In some embodiments, at least one feature has a different cross-sectional shape compared to another feature.

As will be appreciated, the features may be attached to the tubular member in any suitable fashion. For example, the features may be glued, bonded, or otherwise affixed thereto. In such embodiments, the features may not be removable from the tubular member. As will be appreciated, the features also may be integrally formed with the tubular member in some embodiments. In still other embodiments, the feature may be configured to be attachable to the tubular member (e.g., by a clinician) before insertion of the tubular member into the patient’s vasculature.

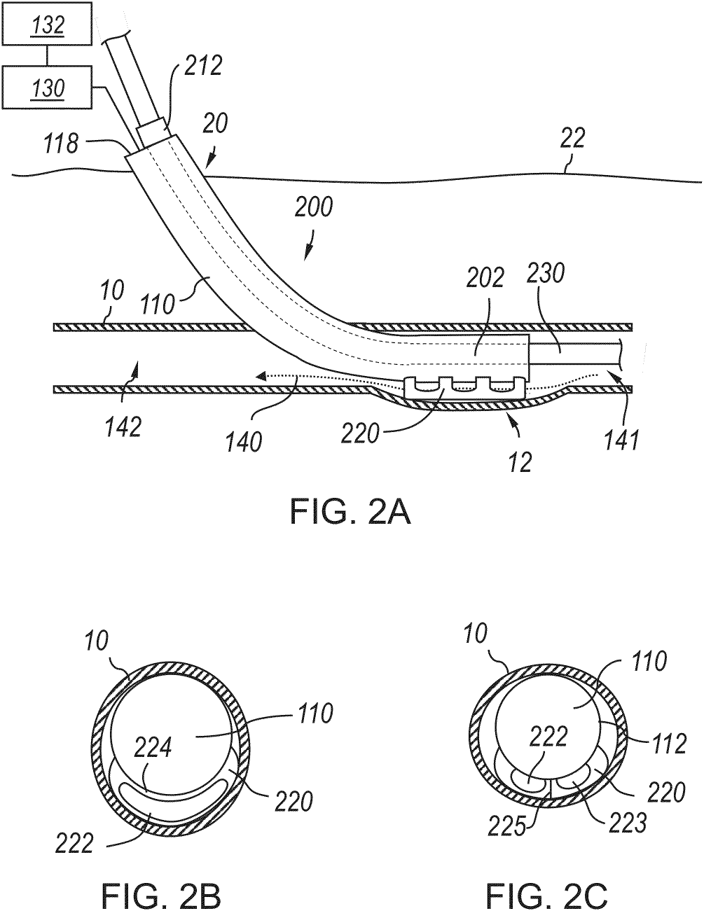

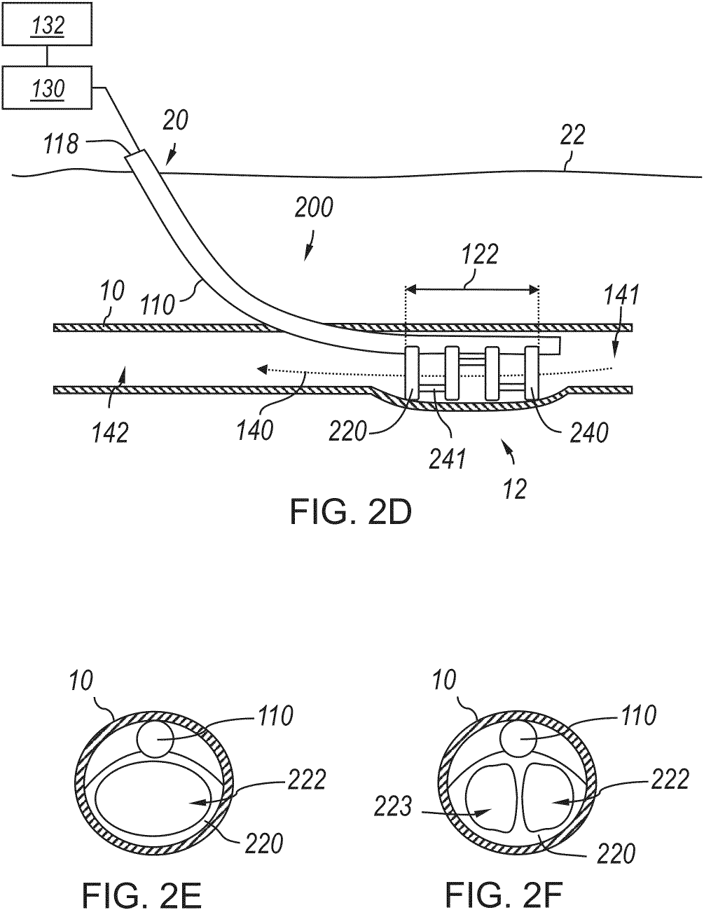

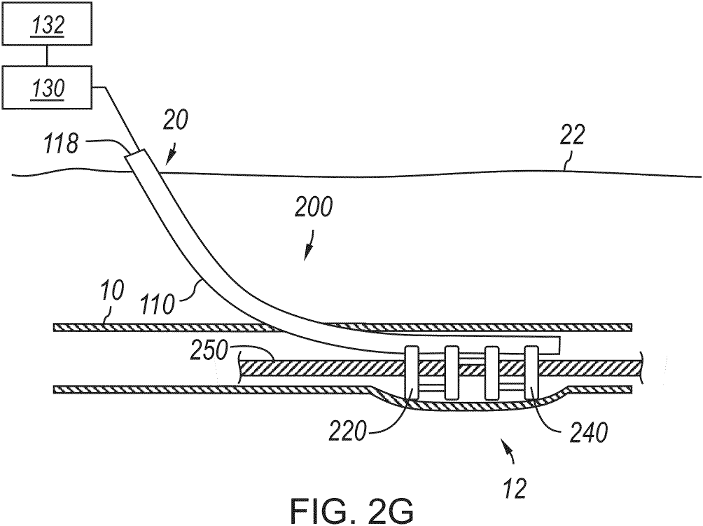

In some embodiments, the radially expanding feature may be configured as a hydrostatic skeleton, as shown in FIGS. 2A-2C. Referring to FIG. 2A, in some embodiments, the device 200 may include a tubular member 110 having a hydrostatic skeleton 220 coupled to the tubular member. As will be appreciated, the hydrostatic skeleton may be attached in a manner similar to that of the features described herein. The hydrostatic skeleton may be comprised of any appropriate flexible material, such a polyurethane or Teflon, which can be inflated to a certain pressure that corresponds to a particular outer diameter. As will be appreciated in view of the above, the hydrostatic skeleton may be connected to a fluid source and a controller which may selectively add and/or remove fluid from the skeleton to selectively expand the hydrostatic skeleton to allow blood flow around the tubular member. As with the above examples, the hydrostatic skeleton may have a collapsed and expanded configuration.

In some embodiments, the hydrostatic skeleton may be configured to be selectively inflated or deflated to create at least a first channel 222 (see FIG. 2B and FIG. 2E) for allowing blood to flow 140 from a point 141 upstream of the hydrostatic skeleton, through the first channel, and to a point 142 downstream of the hydrostatic skeleton. As will be appreciated, similar to the above examples, expansion of the hydrostatic skeleton also may increase a diameter of the patient’s vasculature around the expanded skeleton.

In some embodiments, the maximum diameter (such as a maximum effective diameter) of the first channel may be 1Fr, 3Fr, 5Fr, 7Fr, 9 Fr, 10 Fr, 11 Fr, 12 Fr, 13 Fr, 15 Fr, 17 Fr, 20 Fr, or 23 Fr. In some embodiments, the hydrostatic skeleton may be configured to be selectively inflated or deflated to create at least a first channel 222, and optionally a second channel 223 (see FIGS. 2C and 2F). In some embodiments, the maximum diameter (such as a maximum effective diameter) of the second channel may be 1Fr, 3Fr, 5Fr, 7 Fr, 9 Fr, 10 Fr, 11 Fr, 12 Fr, 13 Fr, 15 Fr, 17 Fr, 20 Fr, or 23 Fr. As will be appreciated, the first and/or second channels may have any suitable diameters. As will be further appreciated, the hydrostatic skeleton may be formed with other suitable numbers of channels. In embodiments having more than one channels, the effective diameter of the first and second channels may be the same, although the diameters also may vary. As will be further appreciated the first and/or second channels may have any suitable shape.

When initially deployed and in the collapsed configuration, the hydrostatic skeleton will have a minimal cross-section. When inflated and in the expanded configuration, the hydrostatic skeleton may have a cross-sectional area that is no greater than a cross-sectional area of the tubular member. In some embodiments, the hydrostatic skeleton may have a cross-sectional area that relatively smaller than a cross-sectional area of the tubular member. Referring to FIG. 2B, a relatively small hydrostatic skeleton is shown with a single channel (e.g., only first channel 222). In other embodiments, the hydrostatic skeleton may have a diameter that is the same as that of the tubular member. In still other embodiments, as illustrated in FIGS. 2D-2F, the hydrostatic skeleton may have a cross-sectional area that larger than that of the tubular member. In FIGS. 2D and 2E, a hydrostatic skeleton is shown with a relatively larger cross-sectional area relative to the tubular member.

Referring to FIG. 2C, the outer surface 112 of the tubular member 110 may form a portion of the first channel 222. Also, as seen, in some embodiments, the skeleton may include plurality of channels, including, e.g., first channel 222 and second channel 223. In some embodiments, the skeleton may include a separator 225 such that at least two channels can be controlled separately. As will be appreciated, in some embodiments, the separator may include a membrane separating the first and second channels. In other embodiments, the separator may be a portion of the skeleton and may itself be expandable. In some embodiments, a cross-sectional area of the first and second channels may be identical. In some embodiments, a cross-sectional area of the first and second channels may be different.

In some embodiments, a diameter of the first channel may increases as the hydrostatic skeleton is inflated.

In some embodiments, a cross-sectional area of the first channel may be controlled separately from the cross-sectional area of the second channel. For example, as seen in FIG. 2C, the membrane or divider 225 may fluidly separate the portion of the skeleton around first channel 222 from the portion around second channel 223, thus allow each section to be controlled separately. In some embodiments, the device simply has two separate hydrostatic skeletons, each with a single channel.

In some embodiments, the tubular member 200 may be configured to slidably receive an additional tubular member 230 (such as a catheter). This may be received, e.g., through a channel or lumen 202 (see e.g., FIG. 2A). In some embodiments, the maximum diameter (such as a maximum effective diameter) of the channel or lumen 202 may be 9 Fr, 10 Fr, 11 Fr, 12 Fr, 13 Fr, 15 Fr, 17 Fr, 20 Fr, or 23 Fr.

In some embodiments, the device may include a valve 212 at a proximal end 118 of the tubular member 110. The valve may be operably coupled to channel or lumen 202. The valve may be configured to receive an additional tubular member 230. In some embodiments, the tubular member 110 and the additional tubular member 230 may be integrally formed.

The hydrostatic skeleton may be coupled at or near the distal end of the tubular member. In some embodiments, the hydrostatic skeleton may extend fully around a circumference of the tubular member. In some embodiments, the hydrostatic skeleton may extend only partially around a circumference of the tubular member.

In some embodiments, there may be only a single hydrostatic skeleton. In some embodiments, there may be a plurality of hydrostatic skeletons.

Referring to FIG. 2D, it is seen that at least a portion of the hydrostatic skeleton 220 may extend along an axial length 122 of the tubular member.

In some embodiments, the hydrostatic skeleton 220 may include a plurality of axially separated rings 240, where each ring may be in contact with the tubular member 110. In some embodiments, each ring may be controlled individually (e.g., selectively expanded via the fluid). In some embodiments, the entire skeleton is controlled as a collective unit. In some embodiments, two or more rings may be separated by a connector 241, which may allow fluid to pass from one ring to a connected ring. In some embodiments, the connector also may maintain a prescribed distance between adjacent rings.

In some embodiments, the rings may form a single channel extending through the hydrostatic skeleton (see, e.g., FIG. 2E). In some embodiments, such as that seen in FIG. 2F, one or more rings may define two channels.

Although shown and described as allowing blood flow through the channels of hydrostatic skeleton, it will be appreciated that the channels may be configured for passage of other devices. For example, referring to FIG. 2G, the hydrostatic skeleton may be configured to allow an additional tubular member 250 to extend at least partially through the hydrostatic skeleton. In some embodiments, the tubular member 250 may extend through the entire hydrostatic skeleton. The additional tubular member may extend through one or more rings 240 of the hydrostatic skeleton. In some embodiments, the tubular member 250 may extend through only one of the channels of the hydrostatic skeleton.

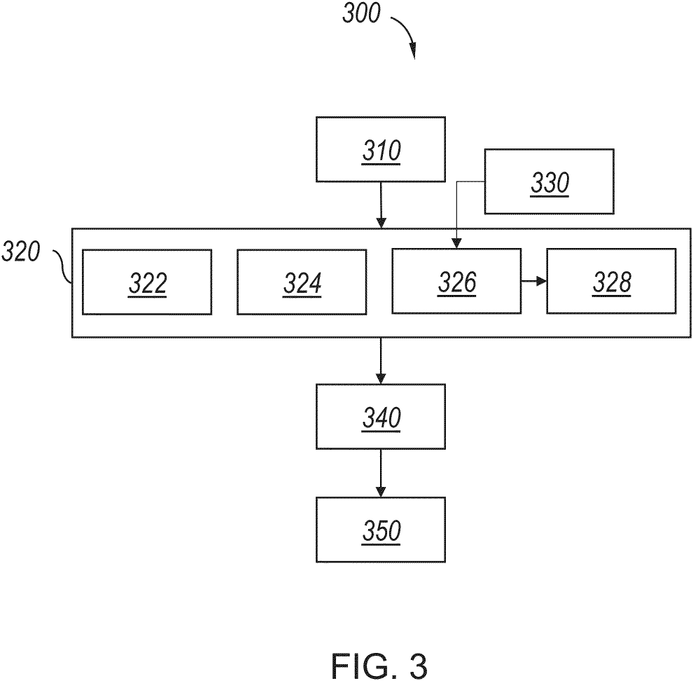

In various aspects, the devices used herein may be used to reduce and/or prevent limb ischemia, as described in the method provided herein. Referring to FIG. 3, the method 300 may include positioning 310 a tubular member within a blood vessel. In some embodiments, the tubular member may allow passage of one or more medical devices, such as a mechanical circulatory support device used to treat the patient. As will be appreciated, in some instances, the tubular member may reduce and/or prevent the flow of blood through the blood vessel in at least one portion of the blood vessel, which may cause ischemia.

The method may therefore also include expanding 320 at least one radially expandable feature from a collapsed configuration to an expanded configuration to expand the portion of the blood vessel and allow blood to flow from a point upstream of the at least one radially expandable feature, around or through the at least one radially expandable feature, and to a point downstream of the at least one radially expandable feature. In some embodiments, the expandable feature may include a hydrostatic skeleton, and the method may include allowing blood to flow through one or more channels of an expanded hydrostatic skeleton.

In some embodiments, expanding from a collapsed configuration to an expanded configuration may include expanding 322 all of the plurality of radially expandable features simultaneously. Expanding from a collapsed configuration to an expanded configuration may also include selectively expanding 324 fewer than all of the plurality of radially expandable features.

In some embodiments, expanding the at least one radially expandable feature may include inflating the at lest one expandable feature by providing a fluid to the expandable feature from a fluid source.

In some embodiments, expanding from a collapsed configuration to an expanded configuration may include receiving 326 information, and then, based on the received information, automatically e 328 from a collapsed configuration to an expanded configuration. For example, referring to FIG. 1A, the disclosed device may include one or more sensors 102 (such as one or more pressure sensors positioned along the device, within the blood vessel). In some embodiments, based on information received from the sensors, the method may include automatically expanding the features from the collapsed configuration to the expanded configuration. In some embodiments, one or more processors may determine that, based on values from the one or more sensors (or from another device, such as a monitoring patch or a mechanical circulatory support device), that an ischemic event is occurring, or may be occurring, and based on that determination, automatically expanding the one or more features from the collapsed configuration to the expanded configuration. As will be understood, the method may include gathering the necessary data to allow such automatic corrections to occur, such as measuring 330 a pressure within the blood vessel, such as upstream and/or downstream from the from the radially expandable feature(s).

Upon completion of a medical procedure or support (e.g., via a device that was inserted into the patient’s vasculature via the tubular member), the method may include removing the tubular member from the patient. In this regard, the method may thereafter include deflating 340 the at least one expandable feature such that the at least one feature returns to the collapsed state. This step may also include ensuring that all of the radially expandable features are in a collapsed configuration. The method may finally include removing 350 the tubular member from the blood vessel and closing the access site.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

What is claimed is:

1. A device, comprising:
a tubular member; and
one or more radially expandable features disposed on an outer surface of the tubular member, the one or more radially expandable features configured to be selectively expanded from a collapsed configuration to an expanded configuration to allow blood within a blood vessel of a patient to flow from a point upstream of the one or more radially expandable features, around and/or through the one or more radially expandable features, and to a point downstream of the one or more radially expandable features.

2. The device according to claim 1, wherein the one or more radially expandable features comprises one or more balloons.

3. The device according to claim 1, wherein the one or more radially expandable features extends along a portion of a length of the tubular member.

4. The device according to claim 1, wherein each radially expandable feature has an identical axial length.

5. The device according to claim 1, wherein at least one of the one or more radially expandable features has a different axial length than another of the one or more radially expandable features.

6. The device according to claim 1, wherein the one or more radially expandable features comprises a plurality of radially expandable features.

7. The device according to claim 6, wherein each of the plurality of radially expandable features is separated circumferentially from an adjacent radially expandable feature by an identical distance.

8. The device according to claim 6, wherein each of the plurality of radially expandable features is separated circumferentially from an adjacent radially expandable feature, and wherein a circumferential separation distance of a first adjacent pair of the plurality of adjacent radially expandable features is different from a circumferential separation distance of a second adjacent pair of the plurality of adj acent radially expandable features.

9. The device according to claim 1, wherein a central axis of each radially expandable features is parallel to a central axis of the tubular member.

10. The device according to claim 1, wherein at least one radially expandable feature is disposed in a helical pattern on the outer surface of the tubular member.

11. The device according to claim 1, wherein at least one radially expandable feature has an oval or circular cross-sectional shape.

12. The device according to claim 1, wherein at least one radially expandable feature has a polygonal cross-sectional shape.

13. The device according to claim 1, wherein each of the at least one radially expandable feature is fluidly connected to a fluid source.

14. The device according to claim 1, wherein each of the at least one radially expandable feature is operably connected to a connector.

15. The device according to claim 1, wherein the at least one radially expandable feature includes a hydrostatic skeleton.

16. A device, comprising:
a tubular member; and
a hydrostatic skeleton coupled to the tubular member, the hydrostatic skeleton configured to be selectively expanded from a collapsed configuration to an expanded configuration to create at least a first channel for allowing blood to flow from a point upstream of the hydrostatic skeleton, through the first channel, and to a point downstream of the hydrostatic skeleton.

17. The device according to claim 16, wherein the hydrostatic skeleton is configured to be selectively inflated or deflated to create at least the first channel and a second channel.

18-26. (canceled)

27. The device according to claim 17, wherein a diameter of the first channel increases when the hydrostatic skeleton is inflated.

28. The device according to claim 17, wherein the hydrostatic skeleton includes a plurality of annular rings.

29. A method, comprising:
positioning a tubular member within a blood vessel, the tubular member having an outer surface and at least one radially expandable member coupled to the outer surface;
expanding the least one radially expandable feature from a collapsed configuration to an expanded configuration to allow blood to flow from a point upstream of the at least one radially expandable feature, around and/or through the at least one radially expandable feature, and to a point downstream of the at least one radially expandable feature.

30-40. (canceled)
